(19) United States
(12) Patent Application Publication
(10) Pub. No.: US 2022/0017839 A1
(43) Pub. Date: Jan. 20, 2022

(54) SYSTEM AND METHOD FOR EXTRACTION OF ESSENTIAL OILS FROM A BOTANICAL BIOMASS

(71) Applicant: Tread Heads, LLC, Sandpoint, ID (US)

(72) Inventor: Bryce W. Powell, Sandpoint, ID (US)

(73) Assignee: Tread Heads, LLC, Sandpoint, ID (US)

(21) Appl. No.: 16/930,853

(22) Filed: Jul. 16, 2020

(51) Int. Cl. C11B 9/02; C07D 311/78; C07C 37/00; B01D 11/02

(52) U.S. Cl. CPC C11B 9/027; C07D 311/78; B01D 11/0211; B01D 11/028; C07C 37/004

(57) ABSTRACT

The separation of essential oils from a botanical biomass can be accomplished in a more effective way and with higher yields using microwaves to extract essential oils by dry or azeotropic distillation and isolating products from the essential oils.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a system and method of extracting essential oils, such as cannabidiol (CBD), from hemp biomass and essential oils from mint. In particular, it relates to extraction and separation utilizing microwave energy.

### Description of Related Art

Extraction of essential oils from a botanical biomass is well known and there are several methods, but all have drawbacks. As an example, cannabinoids are valuable substances found in hemp plants.

Cannabinoids are any of various naturally occurring biologically active, chemical constituents of hemp or cannabis.

Cannabidiol is a phytocannabinoid discovered in 1940. It is one of 113 identified cannabinoids in cannabis plants and accounts for up to 40% of the plant's extract. In 2018, clinical research on cannabidiol included preliminary studies for the treatment of anxiety, cognition, movement disorders, and pain.

Cannabidiol can be taken into the body in multiple ways, including by inhalation of cannabis smoke or vapor, as an aerosol spray into the cheek, and by mouth. It may be supplied as CBD oil containing only CBD as the active ingredient (i.e., no included tetrahydrocannabinol [THC] or terpenes), a full-plant CBD-dominant hemp biomass extract oil, capsules, dried cannabis, or as a prescription liquid solution. CBD does not have the same psychoactivity as THC, and may change the effects of THC in the body if both are present. As of 2018, the mechanism of action for its biological effects has not been determined.

Essential oils are normally extracted from botanical biomass by use of carbon dioxide supercritical fluid extraction (SFE), steam distillation, or use of solvents, such as hydrocarbon solvents. The SFE requirement for high pressure increases the cost compared to conventional extraction, so SFE will only be used where there are significant advantages. Carbon dioxide itself is non-polar, and has somewhat limited dissolving power, so this can create problems with solubility. Food grade modifiers, such as ethanol, can often be used, and can also help in the collection of the extracted material, but reduces some of the benefits of using a solvent, which is gaseous at room temperature. Steam distillation has been around for centuries and is well tested. It is less preferred compared to other methods due to its ability to damage the essential oil extract and alter the chemical properties of the extract. Solvent extraction is more efficient than steam distillation and is also less expensive. However, the solvents utilized in extraction (naptha, petroleum, butane, or propane) are causes for concern because of the toxic effect possibilities with these solvents. Accordingly, there is room for improved methods of extracting essential oils from a botanical biomass.

### BRIEF SUMMARY OF THE INVENTION

The present invention relates to a method for clearly and effectively separating essential oils such as cannabinoid oil from a botanical biomass using a microwave applicator and dry or azeotropic distillation.

Accordingly, in one embodiment, there is a solvent free method for the extraction of essential oils from a botanical biomass comprising:
a) placing the botanical biomass in a microwave applicator;
b) introducing an inert gas into the microwave applicator sufficient to purge oxygen from the microwave applicator;
c) introducing microwave emissions from a microwave transmitter into the microwave applicator, wherein the microwave transmitter is kept at a temperature from about 5° C. to about 25° C. with the temperature in the microwave applicator kept at about 100° C. to about 200° C., until the essential oils from the botanical biomass are vaporized and extracted from the botanical biomass by dry distillation in a low oxygen atmosphere or by azeotropic distillation with the residual water in the botanical biomass;
d) condensing the vaporized essential oils from the microwave applicator to liquid essential oils; and
e) collecting the liquid essential oils and exhausting non-condensable gases by vacuum pump.

### BRIEF DESCRIPTION OF THE DRAWINGS

FIG. 1 shows the relative system components of the method with a hemp biomass, as an example.

FIG. 2 is a flow chart of the method of the present invention with a hemp biomass, as an example.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “solvent free” refers to an essential oil such as hemp (cannabis) concentrate that contains no residual solvents in the full spectrum of the extract. The product is considered safer and superior to products that contain residual solvents, which may or may not be harmful when ingested, depending on the solvent in question. It also refers to there being no solvents other than water used in the initial microwave extraction. In one embodiment, all water in the extraction process comes from the botanical biomass itself. In another embodiment, there is enough added water to prevent charring of the botanical biomass. This is used when there is not enough residual water in the botanical biomass itself. In another embodiment, extraction occurs through dry distillation which involves heating solid materials in a low oxygen atmosphere to produce gaseous byproducts.

As used herein, the term “cannabidiol” refers to cannabidiol (CBD), a naturally occurring compound found in the resinous flower of hemp (cannabis), a plant with a rich history as a medicine going back thousands of years. Both CBD and THC have significant therapeutic attributes. But unlike THC, CBD does not make a person feel “stoned” or intoxicated. It has been implicated in several different types of therapy including as a treatment for depression, anxiety, insomnia, and childhood epilepsy syndromes, such as Dravet syndrome and Lennox-Gestaut syndrome (LGS). CBD can be refined and isolated from cannabinoid oil by short path distillation and chromatography.

As used herein, the term “cannabinoid” refers to any of a group of naturally-occurring, biologically active chemical constituents of hemp or cannabis. 113 distinct cannabinoids have been identified including cannabigerol (CBG), cannabichromene (CBC), and cannabinol (CBN) among others.

As used herein, the term “botanical biomass” refers to all organic material of botanical biomass including flowers, stalks, leaves, roots and/or seeds of the harvested plant. In one embodiment, the “botanical biomass” consists of the group consisting of hemp, mint, citrus, lavender, dill, coffee beans, and crop waste. For collection of oils, the botanical biomass is dried as effectively as possible before further processing. Some naturally occurring residual water can be left in the process to assist in the azeotropic distillation of essential oils.

As used herein, the term “microwave applicator” refers to a device that is designed to heat a material, i.e., botanical biomass, by exposing it to a microwave field in a controlled environment. The microwave field is delivered to the microwave applicator by a microwave transmitter. The temperature of the microwave applicator is kept at about 100° C. to about 200° C. In one embodiment, it is kept at about 165° C.

As used herein, the term “purge oxygen” refers to a system that can be flushed with an inert gas, e.g., nitrogen, to reduce the concentration of oxygen in the system reactor so that combustion and oxidation are reduced. In combustion engineering terms, the admission of inert gas dilutes the oxygen below the limiting oxygen concentration. A low oxygen atmosphere (below about 11%) is necessary in the process to perform dry distillation.

As used herein, the term “microwave emissions” refers to a form of electromagnetic radiation with wavelengths ranging from about one meter to one millimeter; with frequencies between 300 MHz (1 m) and 300 GHz (1 mm). Different sources define different frequency ranges as microwaves; the above broad definition includes both UHF and EHF (millimeter wave) bands. A more common definition in radio-frequency engineering is the range between 1 and 100 GHz (wavelengths between 0.3 m and 3 mm). In all cases, microwaves include the entire SHF band (3 to 30 GHz, or 10 to 1 cm) at minimum. Frequencies in the microwave range are often referred to by their IEEE radar band designations: S, C, X, Kᵤ, K, or Kₐ band, or by similar NATO or EU designations.

As used herein, the term “microwave transmitter” refers to a system that uses a beam of electromagnetic waves in the microwave frequency range to transmit the microwaves between two fixed locations, i.e., between the microwave emissions generator and the microwave applicator, frequently delivered by use of a wave guide. The microwave transmitter is kept at a temperature from about 5° C. to about 25° C. A chiller or heat exchange can be used for all cooling effects needed.

As used herein, the term “essential oils” refers to natural oils typically obtained from plants, e.g., hemp or mint biomass, and having the characteristic fragrance of the plant or other source from which it is extracted. CBD and other cannabinoids are mixed in with the essential oils during extraction, and as such for purposes of this invention, essential oils includes CBD and other cannabinoids. Cannabidiol oil (CBD) is a cannabinoid sourced from the Cannabis sativa genus of plants, usually from industrial hemp plants. The extraction process, in one embodiment, takes about 33 minutes. In one embodiment the extraction takes from about 25 to 35 minutes.

As used herein, the term “azeotropic distillation” refers to distillation involving the presence of a compound that forms an azeotrope with at least one of the components of a liquid mixture, in this case insitu water is used, which can thereby be more readily separated because of the resulting increase in the difference between the volatilities of the components of the mixture.

As used herein, the term “dry distillation” refers to distillation of compounds in a dry condition by heating organic solid materials in a low oxygen atmosphere to produce gaseous products, which may condense into liquids or solids.

In other embodiments, there is a vacuum pump which is used to exhaust non-cannabidiol components of the essential oil, steam, and the like. In another embodiment, microwave emissions are introduced into the applicator via a waveguide. In another embodiment, inert gas (e.g., nitrogen) is heated. In another embodiment, water to the condenser is heated by an industrial water heater.

### DRAWINGS

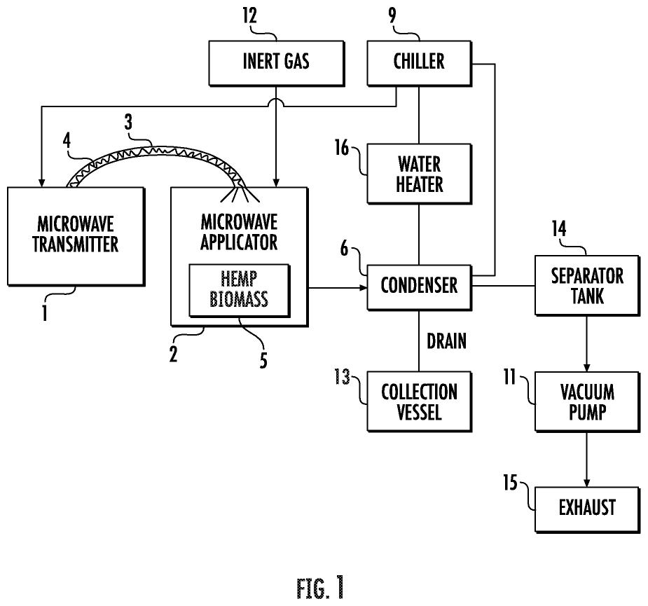

Now referring to the drawings, FIG. 1 is a schematic of the devices used in the present method of extracting essential oils from a botanical biomass using cannabinoids from hemp biomass, as an example. In this embodiment, a microwave transmitter 1 transmits microwave emissions 4 via a waveguide 3 to microwave applicator 2. The temperature of the microwave transmitter 1 is controlled by the chiller 9. The hemp biomass 5 is placed in the microwave applicator 2 and an inert gas 12 like nitrogen, is used to purge the inside of the microwave applicator 2.

Essential oils are gasified by dry or azeotropic distillation and enter a condenser 6, cooled by a chiller 9. A water heater 16 controls temperature of water to the condenser 6. Condensed liquid oils are sent to a collection vessel 13, which captures full spectrum essential oils including CBD and other cannabinoids. Waste collection occurs in a separator tank 14, and is powered by a vacuum pump 11, with exhaust 15.

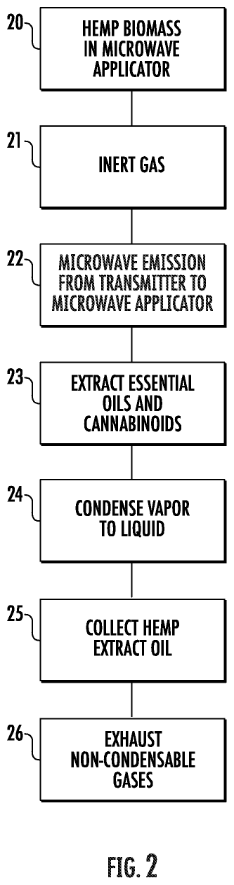

FIG. 2 is a flow chart of the process of the present invention using hemp biomass, as an example. Hemp biomass is placed in the microwave applicator 20. An inert gas 21 is used to purge the microwave applicator. The microwave emissions from the microwave transmitter are transferred to the microwave applicator 22. The essential oils including CBD and other cannabinoids are extracted 23 via dry or azeotropic distillation, condensed 24 and captured in a collection tank 25. Non-condensable gases are removed via exhaust 26.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A solvent free method for the extraction of essential oils from a botanical biomass comprising:
a) placing the botanical biomass in a microwave applicator;
b) introducing an inert gas into the microwave applicator sufficient to purge oxygen from the microwave applicator;
c) introducing microwave emissions from a microwave transmitter into the microwave applicator, wherein the microwave transmitter is kept at a temperature from about 5° C. to about 25° C. with the temperature in the microwave applicator kept at about 100° C. to about 200° C., until the essential oils from the botanical biomass are vaporized and extracted from the botanical biomass by dry distillation in a low oxygen atmosphere or by azeotropic distillation with the residual water in the botanical biomass;
d) condensing the vaporized essential oils from the microwave applicator to liquid essential oils; and
e) collecting the liquid essential oils and exhausting non-condensable gases by vacuum pump.

2. The solvent free method according to claim 1 which includes using a chiller to control temperatures in the method.

3. The solvent free method according to claim 1 wherein a pump is used to exhaust non-condensable gas.

4. The solvent free method according to claim 1 wherein the microwave transmitter introduces microwave emissions to the microwave applicator via a waveguide.

5. The solvent free method according to claim 1 wherein the botanical biomass is extracted for about 25 to 35 minutes.

6. The solvent free method according to claim 1 wherein the botanical biomass is extracted for about 33 minutes.

7. The solvent free method according to claim 1 wherein there is water added to the botanical biomass to help prevent charring the botanical biomass.

8. The solvent free method according to claim 1 wherein the botanical biomass is kept at about 165° C. during extraction.

9. The solvent free method according to claim 1 wherein the inert gas is nitrogen.

10. The solvent free method according to claim 1 wherein the inert gas is heated.

11. The solvent free method according to claim 1 wherein a water heater is used to control temperatures in the condenser.

12. The solvent free method according to claim 1 wherein the botanical biomass is selected from the group consisting of hemp, mint, citrus, lavender, dill, coffee beans, and crop waste.

13. The solvent free method according to claim 12 wherein cannabinoids are isolated from the hemp biomass essential oils.
